(19) United States
(12) Patent Application Publication
(10) Pub. No.: US 2022/0362063 A1
(43) Pub. Date: Nov. 17, 2022

(54) COOLING COHESIVE TAPE

(71) Applicant: Shawsheen Rubber Co, Inc., Andover, MA (US)

(72) Inventor: Gary Litman, Framingham, MA (US)

(21) Appl. No.: 17/742,885

(22) Filed: May 12, 2022

Related U.S. Application Data
(60) Provisional application No. 63/189,369, filed on May 17, 2021.

(51) Int. Cl. A61F 13/02; C08L 23/12; A61F 13/00

(52) U.S. Cl. CPC A61F 13/0273; C08L 23/12; A61F 13/00063; A61F 2013/00659; A61F 2013/00604

(57) ABSTRACT

An athletic tape product for securing or supporting a human or animal body part is provided that includes a substrate impregnated with an aqueous elastomer dispersion comprising cooling agents.

### CROSS-REFERENCE TO RELATED APPLICATION

This application claims the benefit of an earlier filing date from U.S. Provisional Application Ser. No. 63/189,369 filed May 17, 2021, the entire disclosure of which is incorporated herein by reference.

### BACKGROUND

The invention is directed to cohesive products, and more particularly to cohesive tapes, in which the cohesive tape comprises cooling agents.

Cohesive athletic tapes are used for a variety of purposes. Rigid cohesive tapes are used to immobilize injuries, while cohesive stretch tapes are used to support joints while allowing them mobility to flex. Cohesive tapes are an ideal way of providing stabilization and support as they stick or bond to themselves when applied to a wearer, but do not adhere to the skin, hair, or fur of the wearer. Cohesive tapes are typically produced by a saturation lamination of multiple web materials with a cohesive rubber emulsion.

A drawback with currently known athletic cohesive tapes is that they do not provide a means for cooling the skin to which the tape is applied. NASA has documented testing that individuals physically perform better when cooling is used to prevent overheating. When athletes are performing strenuously they generate heat. Any temperature reduction during these times results in better physical performance.

A cohesive tape having cooling properties is taught in U.S. Patent Application Publication No. 20190001016 (“'016”). The tape taught in '016 is formed with an aqueous alcohol solution that cools the surrounding air via evaporation. Once the alcohol has evaporated, the tape becomes dry and no longer produces a cooling effect. This occurs after about 15 minutes. The tape taught in '016 also requires special packaging to keep it wet during shipping, distribution, and between uses, which makes it difficult to package and which further reduces the shelf life of the tape. The tape taught in '016 also tends to unravel and fall off after it is dried.

Therefore, what is needed is a cohesive tape that has a cooling effect to a user when the cohesive tape is applied to the skin of the user. Further needed is a cohesive tape that has a cooling effect and which can be “reactivated” once the cooling effect has been reduced. Further needed is a cohesive tape that has a cooling effect and which is cost effective to manufacture and which is efficiently packaged and which has a longer shelf-time both during and after the manufacturing process.

### SUMMARY

The need in the art is addressed by the cohesive tape of the present invention which is formed to have a cooling effect when worn by a user. It is noted herein that, although the cohesive tape of the present invention is particularly advantageous when worn by an individual while engaged in an athletic event such as basketball, track, tennis, baseball, football, and the like, the invention is not so limited, but includes a cohesive tape worn whenever it is desired or beneficial to have both the support offered by the tape and the cooling effect.

In a most general embodiment, the cohesive tape includes a substrate impregnated with an aqueous based emulsion comprising one or more of an endothermic reaction agent and a thermally conductive member. In an exemplary embodiment, the endothermic reaction agent comprises one or more of menthol and micro encapsulated phase change particles, and the thermally conductive member comprises a ceramic filler, wherein an especially preferred ceramic filler comprises aluminum tri-hydroxide.

The above and other features of the invention, including various novel details of construction and combinations of parts, will now be more particularly described with reference to the accompanying drawings. It will be understood that the particular devices and method embodying the invention are shown by way of illustration only and not as limitations of the invention. The principles and features of this invention may be employed in various and numerous embodiments without departing from the scope of the invention.

### BRIEF DESCRIPTION OF THE DRAWINGS

The following descriptions should not be considered limiting in any way. With reference to the accompanying drawings, like elements are numbered alike:

FIG. 1 is a cross-sectional view of a first embodiment of the invention, in which a multi-layered substrate has been impregnated with a synthetic cohesive, water-based elastomer.

FIG. 2 is a top view of the embodiment of FIG. 1.

FIG. 3 is a cross-sectional view of a second embodiment comprising a knitted substrate and a synthetic cohesive, water-based elastomer that is deposited on the opposite sides of a single layer of knitted substrate, such as by spraying or coating.

### DETAILED DESCRIPTION

Illustrative embodiments and exemplary applications will now be described with reference to the accompanying drawings to disclose the advantageous teachings of the present invention.

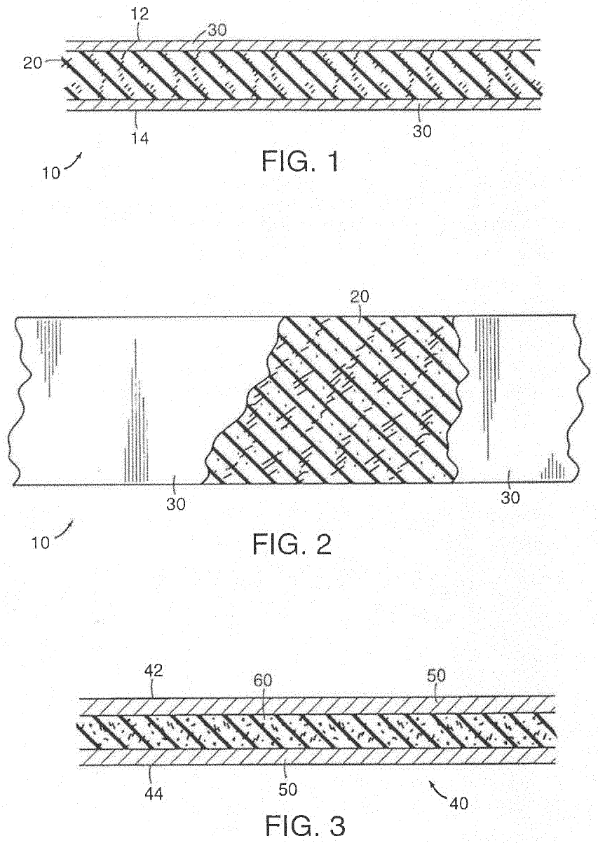

Referring more particularly to the drawings, FIG. 1 is a cross-sectional view of a tape, generally designated 10, in which a substrate 20 has been impregnated through the thickness of the substrate with a synthetic, cohesive, water-based elastomer 30. Substrate 20 may include a plurality of longitudinally-extending elastic threads or yarns sandwiched between a layer of a warp-knit (weft-insertion) fabric and a layer of a non-woven fabric. In the present embodiment, cohesive elastomer 30 bonds the three layers together. The elastomer 30 also extends fully through the thickness of the substrate, so that the top and bottom surfaces of the overall tape 10 are defined by synthetic elastomer 30.

FIG. 2 is a top view of the tape 20 of FIG. 1, cut away so as to better show each of the three layers of the substrate 20.

FIG. 3 is a cross-sectional view of a second tape, generally designated 40, in which a synthetic, cohesive, water-based elastomer 50 has been coated or sprayed onto the opposite sides of a single layer of a woven or knitted substrate 60. As with the embodiment of FIG. 1, it will be recognized that the top and bottom surfaces 42, 44 of the overall tape 40, are defined by the synthetic elastomer. Since the substrate is only a single layer, however, the elastomer is not required to bond a multi-layer structure together and need not extend through the thickness of the substrate.

Because the synthetic elastomers 30, 50 of FIGS. 1 and 3, respectively, are cohesive, it will be recognized that the outer surfaces of the tapes 10, 40 of FIGS. 1 and 3, respectively, will stick to each other, e.g., the top surface 12 of tape 10 will stick to bottom surface 14 when the tape is wrapped around, for example, a user's ankle; and the top surface 42 of tape 40 will similarly stick to bottom surface 44. However, because the synthetic elastomers are cohesive, rather than pressure sensitive, the surfaces of tapes 10, 40 will not stick (at least to any significant degree) to other surfaces or materials.

It will be recognized that substrates 20, 60 may be made of any of a wide range of materials, and may have a wide range of structures. For example, any of the one or more layers of a substrate may be, for example, a woven, knitted, warp-knit (weft-insertion) or non-woven fabric, or paper. It may also be a surface-treated polymeric, such as a sheet of linear, low-density polyethylene (“LLDPE”) or linear, low-density polypropylene (“LLDPP”), one or more surfaces of which has been treated to insure adhesion to the elastomeric cohesive. Similarly, the substrate structure may be elasticized, either as described above with reference to FIG. 1, by knitting or weaving elastic threads into one or more of the layers, or by knitting or sewing elastomeric threads through a single or multi-layer substrate.

In certain embodiments in which the cohesive product of the present invention is a tape, the substrate typically will comprise a woven, knitted, or warp-knit (weft insertion) fabric, or a non-woven fabric such as a non-woven scrim, of either natural or synthetic fiber. In one embodiment in this aspect, the substrate comprises a single layer of a non-woven fabric wherein threads are knitted through the fabric and a synthetic cohesive, water-based elastomer is deposited on opposite sides of the fabric by, for instance, spraying or coating. In a preferred tape, the substrates 20, 60 comprise polypropylene, nylon or polyester, wherein polypropylene is especially preferred.

In another embodiment of this aspect, the substrate of the tape comprises a first and a second layer of non-woven fabric and a third layer which is elastic in a direction extending longitudinally of the tape, said third layer being in between the first and second layers of non-woven fabric.

In a further embodiment, the substrate of the tape comprises: a first layer of warp-knitted (weft insertion) fabric oriented with the knit yarns extending longitudinally of the tape; a second layer of a non-woven fabric; and a third layer which is elastic in a direction extending longitudinally of the tape, the third layer being between said first and second layers.

Examples of polymeric rubber bases include one or more of styrene-isoprene-styrene polymers, styrene-olefin-styrene polymers including styrene-ethylene/propylene-styrene polymers, polyisobutylene, styrene-butadiene-styrene polymers, polyisoprene, polybutadiene, natural rubber, silicone rubber, acrylonitrile rubber, nitrile rubber, polyurethane rubber, polyisobutylene rubber, butyl rubber, halobutyl rubber including bromobutyl rubber, butadiene-acrylonitrile rubber, polychloroprene, and styrene-butadiene rubber.

Elastomer 30 comprises a water-based emulsion of a synthetic water-based elastomer where one or more tackifiers are added in an amount that enhances the cohesive property of the elastomer by disruption of the crystalline structure and to maintain the cohesive material in the desired partial polycrystalline state. More specifically, in the practice of the present invention, the synthetic cohesive end product is typically made by:

(1) combining a synthetic, inherently crystalline elastomer with at least one tackifying agent to produce an emulsion of the elastomer and tackifying agent(s)
(2) adding at least one of an endothermic reaction agent and a thermally conductive agent to the emulsion;
(3) providing a substrate of a desired structure; and
(4) treating the substrate with the emulsion such that the emulsion defines at least one outer surface of the product.

The emulsion is applied to the substrate (typically by saturating the substrate with the emulsion or coating the emulsion onto the opposite sides of the substrate), and the structure is then dried to produce the cooling cohesive tape.

Exemplary polymers possess crystalline properties similar to natural rubber. Particularly preferred polymers include water-based polychloroprene emulsions such as poly-2-chloro, 1-4 butadiene and certain water-based polyurethanes, that are inherently capable of crystallization, such as, e.g., polyester polyurethane and polycaprolactone polyurethane.

Exemplary tackifiers include, for example, esters of abietic acid (rosin esters), certain low-molecular weight hydrocarbon resins usually referred to as C₅-C₉ polymers, polymers with low glass transition temperatures such as some acrylic polymers and some butadiene-styrene copolymers, and certain monomeric plasticizers. The tackifiers used to produce cohesive forms of these synthetic elastomeric materials are of the same type used in connection with natural rubber, although the amount(s) of any particular tackifier(s) used to form a stable cohesive will vary within empirically defined limits.

The adhesive or cohesive coating material may be coated at a weight basis of about 40 g/m² to about 150 g/m², preferably from about 50 g/m² to about 100 g/m², wherein 50 g/m² is especially preferred.

The cohesive material may be applied as an aqueous emulsion to both major surfaces of the substrate using, e.g., a dip dank and nip roller apparatus. Alternatively, the cohesive material may be applied to both major surfaces of the substrate as an aqueous emulsion by spraying or coating and drying. In certain embodiments, the cohesive material may be applied to both major surfaces of the substrate as a viscid mass, coatable syrup, or sprayable liquid and spread with rollers or spreader blades.

In preferred practices of the invention, the inherently crystalline, water-based, synthetic elastomer is preferably polychloroprene, such as DuPont NEOPRENE LTX-654, and the tackifying resins used to arrest it in the desired polycrystalline state are one or more of a rosin ester derivative, a petroleum derivative, a hydrocarbon resin, an acrylic polymer, a butadiene-based polymer or a combination of one or more types such as rosin ester/hydrocarbon resin.

In general, any compatible conventional tackifier resin or mixture of such resins may be used. These include hydrocarbon resins, rosin and rosin derivatives, polyterpenes and other tackifiers. The adhesive substance may also include a tackifier. Tackifiers, are generally hydrocarbon resins, wood resins, rosins, rosin derivatives, and the like. It is contemplated that any tackifier known by those of skill in the art to be compatible with elastomeric polymer compositions may be used with the present embodiment of the invention.

As used in the present invention, the terms “tackifier” and “tackifying agent” herein refer to a class of thermoplastic polymers used to affect the characteristics of a finished polymeric product and includes the tackifying resins listed above, naturally occurring rosins, rosin esters, and plasticizers. As used in the present invention, the term “rosin” as used herein refers to a naturally occurring material extracted from stumps of pine trees whose principal component is abietic acid. The term “rosin ester” as used herein, refers to the carboxyl group of abietic acid which has been esterified with aromatic and aliphatic alcohols. The term “hydrocarbon resins” as used herein refers to lower-molecular-weight thermoplastic polymers derived from cracked petroleum distillates, terpene fractions, coal tar, and a variety of pure monomers. Although a single tackifying resin can be used, blends of two or more with different melting points (and molecular weights) have been found to produce cohesive products with better final properties. In some circumstances, plasticizers may be used in lieu of one or more tackifier resins. Synthetic elastomers such as NEOPRENE LTX-654 and tackifying agents are commercially available in dispersion and emulsion forms.

When compounding the elastomer and tackifiers, there exists for each elastomer a “window” of compounding in which the structure of the polychloroprene or other elastomer is crystalline, and within which the degree of crystallinity can be modified so that the material has cohesive properties. The extent of the “window” varies depending on the particular elastomer, and is determined empirically. At one extreme of the “window,” the elastomer becomes non-cohesive, and at the other extreme, it becomes pressure-sensitive. The state of the material within its “window” depends on the extent to which the polycrystalline structure of the polychloroprene or other elastomer is disrupted, and can be varied using different amounts and types of tackifying agents. For any particularly water-based inherently crystalline, synthetic elastomer, the amount and type of tackifier required to arrest the elastomer in a partially crystalline, cohesive state is empirically determined, using tackifiers and protocols similar to those long employed in the production of cohesive natural rubber latex materials and known to one of skill in the art.

When applied to a substrate so that it defines the outer surfaces of a product, a water-based, synthetic inherently crystalline elastomer to which an effective amount of tackifier has been added produces a cohesive product which will adhere to itself, but not (at least to any significant degree) to other substrates.

The emulsion further comprises a thermal conductor agent which conducts heat from the body out to the atmosphere if the ambient temperature is below body temperature. An exemplary thermal conductor agent comprises a ceramic filler. Ceramic fillers have been found to be particularly beneficial to the application of the inventive cohesive tape as the crepe texture of the tape increases the surface area of the ceramic filler, thereby, creating a heat sink which, therefore, allows for a cooling effect. Exemplary ceramic fillers include, e.g., one or more of aluminum tri-hydroxide, magnesium oxide, aluminum oxide, boron nitride, titanium diboride, and the like. In an especially preferred embodiment, the ceramic filler comprises aluminum tri-hydroxide.

The amount of ceramic filler included in the emulsion will need to balance the factors of flexibility or stretch, cohesiveness, and thermal conductivity. The greater the amount of ceramic filler in the emulsion, the greater will be the thermal conductivity, and hence, the cooling effect, but the cohesiveness and the flexibility of the tape may be compromised. Ideal cooling, flexibility, and cohesion of the inventive tape has been found when the ceramic filler does not exceed up to about 100 parts per hundred by weight (“pph”) to polymer solids.

The emulsion further comprises one or more of an endothermic reaction agent, wherein an exemplary endothermic reaction inducing agent comprises menthol. Menthol is a particularly preferred agent as it sublimes at room temperature. The menthol creates endothermic cooling via diffusion over time. In an exemplary embodiment, the emulsion comprises a concentration of menthol that does not exceed up to about 9 wt %, wherein wt % is based upon the dry weight of the cohesive polymer.

Another exemplary endothermic reaction agent comprises micro encapsulated phase change particles (“MEPC particles”), wherein exemplary MEPC particles include those sold by Microtek Laboratories, Inc., wherein an especially preferred MEPC particle is sold under the name PCM 28 by Microtek Laboratories, Inc. MEPC particles are microparticles of a low temperature melting hydrocarbon that are encapsulated in a polymer balloon. The particles store and release large amounts of energy by undergoing a phase change between its liquid and solid states. Melting of the wax is endothermic and generates cooling. As a MEPC particle cycles between the liquid and solid states, it holds the temperature around its particular melt point. Because the wax is encapsulated, the particle can cool and re-solidify and re-melt repeatedly, thereby providing cooling benefits even upon repeated use of the cohesive tape. The endothermic reaction of the MEPC particles happens upon initial touch of the tape by a user, thereby, providing a quick burst of coolness. In an exemplary embodiment, the MEPC particles are selected so that they have a melting point between 27 degrees Celsius to about 30 degrees Celsius, wherein a 28 degree Celsius melt temperature MEPC particle is preferred.

In an exemplary embodiment, the emulsion comprises a concentration of MEPC particles of up to about 60 pph based on the cohesive polymer solids.

An exemplary method of forming an exemplary cohesive tape comprises compounding tackifier resins into polychloroprene in an aqueous emulsion. Using a paddle mixer, menthol, aluminum tri-hydroxide, and MEPC particles, in powder form, are mixed into the emulsion. In an exemplary embodiment, the emulsion comprises about 40-65% solids of polychloroprene, about 35-75 pph of the tackifiers, about 4-9% menthol, about 40-80 pph aluminum tri-hydroxide, and about 20-60 pph MEPC particles.

Mixing may comprise use of an air motor agitator wherein the speed of the agitator is set to create a small vortex in the forming emulsion and the components are mixed in this way for about 15 minutes.

In an exemplary embodiment, the substrate is saturated by dip and squeeze coating a 50-200 g/m² dry coating of about 50-60% solids emulsion onto a composite web of about 10-20 g/m² basis weight.

The saturated substrate is then dried in a 3 zone infrared oven and then through a web path of wrapping around steam heated rolls. In an exemplary embodiment, the infrared oven is set at maximum power for about 40 seconds. In an exemplary embodiment, the steam heated rolls are about 200 degrees Fahrenheit and the saturated substrate is exposed to the steam heated rolls for about 120 seconds to thereby form a master roll. In an exemplary embodiment, the master roll comprises about 40-65% solids of polychloroprene, about 35-75 pph of the tackifiers, about 4-9% menthol, about 40-80 pph aluminum tri-hydroxide, and about 20-60 pph MEPC particles.

If not immediately ready for logging and rolling, the master roll may be stretch wrapped to seal in the menthol. The master roll can be stored for up to about 12 months before subsequent converting into log rolls and slit rolls. Once ready for logging and slitting, the master roll may be unwrapped in front of logging into approximately 5 yard rolls. Preferably, then the logs are immediately single knife slit to about 2 inch widths and flow wrap packaged as quickly as possible so as to avoid the loss of menthol through evaporation.

The use of the terms “a” and “an” and “the” and similar referents in the context of describing the invention (especially in the context of the following claims) are to be construed to cover both the singular and the plural, unless otherwise indicated herein or clearly contradicted by context. The conjunction “or” when used with a list of at least two terms is intended to mean any term or combination of terms. Further, it should be noted that the terms “first,” “second,” and the like herein do not denote any order, quantity, or importance, but rather are used to distinguish one element from another. The terms “including” and “having” and the like are intended to be inclusive such that there may be additional elements other than the elements listed. The terms “about”, “substantially” and “generally” are intended to include a non-significant degree of variation such as with a degree of error associated with measurement of the particular quantity based upon the equipment available at the time of filing the application. For example, “about” and/or “substantially” and/or “generally” can include a range of ±5% of a given value.

The disclosure illustratively disclosed herein may be practiced in the absence of any element which is not specifically disclosed herein.

While the present invention is described herein with reference to illustrative embodiments for particular applications, it should be understood that the invention is not limited thereto. Those having ordinary skill in the art and access to the teachings provided herein will recognize additional modifications, applications, and embodiments within the scope thereof and additional fields in which the present invention would be of significant utility.

What is claimed:

1. A cohesive tape for applying stabilization and support to a biological body part comprising:
a substrate layer having a first side and a second side; and
a cohesive elastomer secured to the first side and the second side, the cohesive elastomer comprising an emulsion, the emulsion comprising at least one of an endothermic reaction agent or a thermally conductive agent, wherein the first side adheres to the second side by contact while the first side and the second side do not adhere to the biological body part due to the cohesive elastomer.

2. The cohesive tape according to claim 1, wherein the substrate layer comprises a single substrate layer wherein the cohesive elastomer does not extend through a thickness of the single substrate layer.

3. The cohesive tape according to claim 1, wherein the substrate comprises a plurality of layers.

4. The cohesive tape according to claim 3, wherein the plurality of layers is impregnated with the cohesive elastomer.

5. The cohesive tape according to claim 3, wherein the substrate comprises a first layer, a second layer, and a third layer, wherein the first layer and the second layer each comprise a non-woven fabric and the third layer comprises a material elastic in a longitudinal direction and is disposed between the first layer and the second layer.

6. The cohesive tape according to claim 3, wherein the substrate comprises a first layer, a second layer, and a third layer, wherein the first layer comprises a warp-knitted fabric oriented with knit yarns extending longitudinally with the tape, the second layer comprises a non-woven fabric, and the third layer comprises a material elastic in a longitudinal direction with the tape and is disposed between the first layer and the second layer.

7. The cohesive tape according to claim 1, wherein the substrate comprises at least one of polypropylene, nylon, or polyester.

8. The cohesive tape according to claim 1, wherein the emulsion is water-based and comprises a synthetic water-based elastomer comprising one or more tackifiers resulting in the emulsion having a disrupted crystalline structure to enhance cohesiveness of the cohesive tape.

9. The cohesive tape according to claim 8, wherein the one or more tackifiers comprise at least one of a hydrocarbon resin, wood resin, rosin, or rosin derivative.

10. The cohesive tape according to claim 9, wherein the one or more tackifiers comprises a blend of two or more tackifiers.

11. The cohesive tape according to claim 1, wherein the thermally conductive agent comprises a ceramic filler.

12. The cohesive tape according to claim 11, wherein the ceramic filler comprises at least one or more of aluminum tri-hydroxide, magnesium oxide, aluminum oxide, boron nitride, or titanium diboride.

13. The cohesive tape according to claim 11, wherein the ceramic filler does not exceed up to about 100 parts per hundred by weight (“pph”) to polymer solids.

14. The cohesive tape according to claim 1, wherein the endothermic reaction agent comprises at least one of menthol or micro encapsulated phase change (MEPC) particles.

15. The cohesive tape according to claim 14, wherein the emulsion comprises about 40-65% solids of polychloroprene, about 35-75 pph of tackifiers, about 4-9% menthol, about 40-80 pph aluminum tri-hydroxide, and about 20-60 pph MEPC particles.

16. A method for applying stabilization and support to a biological body part,
wrapping the biological body part with a cohesive tape, the cohesive tape comprising (i) a substrate layer having a first side and a second side and (ii) a cohesive elastomer secured to the first side and the second side, the cohesive elastomer comprising an emulsion, the emulsion comprising at least one of an endothermic reaction agent or a thermally conductive agent;
adhering the first side to the second side by contact, wherein the first side and the second side do not adhere to the biological body part due to the cohesive elastomer; and
cooling the biological body part using the at least one of the endothermic reaction agent or the thermally conductive agent.

17. The method according to claim 16, further comprising removing the cohesive tape from the biological body part by separating the first side from the second side without pulling on the biological body part.
